(19) United States
(12) Patent Application Publication
(10) Pub. No.: US 2024/0293633 A1
(43) Pub. Date: Sep. 5, 2024

(54) LARYNGEAL TUBE

(71) Applicant: Imam Abdulrahman Bin Faisal University, Dammam (SA)

(72) Inventor: Abdulrahman Athwan ALQARNI, Damman (SA)

(73) Assignee: Imam Abdulrahman Bin Faisal University, Dammam (SA)

(21) Appl. No.: 18/177,447

(22) Filed: Mar. 2, 2023

(51) Int. Cl. A61M 16/04

(52) U.S. Cl. CPC A61M 16/0486; A61M 16/0463; A61M 16/0409; A61M 16/0415; A61M 16/0443; A61M 16/0459; A61M 16/0479; A61M 2205/32

(57) ABSTRACT

A laryngeal tube with a self-sealing valve, a pilot balloon, a cuff inflation line, a suction port, and a radiopaque cover. The laryngeal tube further includes a proximal cuff for securing a pharyngal area of a patient and a distal cuff for preventing oxygen escape into a stomach of the patient. The laryngeal tube further includes a suction lumen configured to reduce a risk of aspiration in a trachea of the patient and a pharyngal lumen configured to provide ventilation capacities. The self-sealing valve surrounds a periphery of the cuff inflation line. The suction port, the cuff inflation line, the self-sealing valve, and the pilot balloon all extend through an interior of the radiopaque cover, which connects the pharyngal lumen to the proximal cuff.

### TECHNICAL FIELD

The present disclosure is directed to a medical device used for an intubation procedure, and particularly, to a laryngeal tube used for providing ventilation to a patient during respiratory distress.

### DESCRIPTION OF RELATED ART

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Laryngeal tubes with 100% blind insertion techniques are used in pre-hospital environments, wards, operating rooms, and post-operative care units to provide ventilation to a patient during respiratory distress. Laryngeal tubes are also used in emergency situations when intubation or ventilation is not possible. Laryngeal tubes function to secure a patient's airway during anaesthesia and cardiopulmonary resuscitation. Laryngeal tubes generally include inflatable cuffs for blocking the oesophagus and for blocking the pharynx such that a fluid communication is established from the patient's mouth to the larynx.

U.S. Pat. No. 5,499,625A describes an airway having two lumens, an inflatable distal cuff, and a pharyngal cuff. The airway includes an air outlet located between the cuffs to communicate with the lumen having a closed distal end. The airway further includes an X-ray opaque stripe and a monitoring device. The multiple elements make the structure of the airway complex, thereby the handling of the airway during intubation procedure becomes difficult.

Accordingly, it is one object of the present disclosure to provide a low cost and an improved laryngeal tube that is more convenient to handle during the intubation procedure. It is also an object of the present disclosure to develop a laryngeal tube that can be fabricated with less complexity.

### SUMMARY

In an exemplary embodiment, a laryngeal tube is described. The laryngeal tube includes a self-sealing valve, a pilot balloon, a cuff inflation line, a suction lumen and a pharyngal lumen, a suction port, a radiopaque cover, a proximal cuff, and a distal cuff. The laryngeal tube includes a first self-sealing valve, a first pilot balloon, and a first cuff inflation line are collected into a first tube. The laryngeal tube includes a second self-sealing valve, a second pilot balloon, and a second cuff inflation line are collected into a second tube. The proximal cuff is configured to secure a pharyngal area of a patient and the distal cuff is configured to prevent oxygen escape into a stomach of the patient. The self-sealing valve surrounds a periphery of the cuff inflation line. The suction port, the cuff inflation line, the self-sealing valve, and the pilot balloon all extend through an interior of the radiopaque cover. The radiopaque cover connects the pharyngal lumen to the proximal cuff. The suction lumen is configured to reduce a risk of aspiration in a trachea of the patient and the pharyngal lumen is configured to provide ventilation to a patient.

In some embodiments, the cuff inflation line is made of polyvinyl chloride (PVC).

In some embodiments, the suction lumen is fixedly attached to an adapter.

In some embodiments, the adapter has a length in a longest dimension of from 10 mm to 50 mm.

In some embodiments, the radiopaque cover comprises at least eight pharyngal perforations.

In some embodiments, the proximal cuff operates at a pressure of from 20 and 50 cm H₂O to properly secure the pharyngal area of the patient.

In some embodiments, the distal cuff operates at a pressure of from 20 and 60 cm H₂O to prevent oxygen escape into the stomach of the patient.

In some embodiments, the proximal cuff accommodates a volume of fluid ranging from 60 milliliters (mL) to 100 mL.

In some embodiments, the distal cuff accommodates a volume of fluid ranging from 60 milliliters (mL) to 80 mL.

In some embodiments, the suction lumen has a first diameter from a distal end of the suction lumen to a first pilot balloon and a second diameter from a proximal end of the suction lumen to the first pilot balloon.

In some embodiments, the pharyngal lumen has a first diameter from a distal end of the pharyngal lumen to a second pilot balloon and a second diameter from a proximal end of the pharyngal lumen to the second pilot balloon. The second diameter is less than the first diameter and the second diameter provides the pharyngal lumen volume of from 50% to 70% the volume of the second pilot balloon.

In some embodiments, the adapter is fixedly attached to the suction lumen by the suction port.

In some embodiments, the suction lumen is fixedly attached to a suction connector or a jet ventilation tube.

In some embodiments, an inner diameter of the radiopaque cover is from 1.1 to 1.4 times greater than an outer diameter of the suction port.

In some embodiments, an inner diameter of the radiopaque cover is from 1.1 to 1.4 times greater than an outer diameter of the cuff inflation line.

In some embodiments, an inner diameter of the radiopaque cover is from 1.1 to 1.4 times greater than an outer diameter of the self-sealing valve.

In some embodiments, an inner diameter of the radiopaque cover is from 1.2 to 1.5 times greater than an outer diameter of the pilot balloon.

In some embodiments, the proximal cuff further comprises a proximal cuff seal.

In some embodiments, the distal cuff further comprises a distal cuff seal.

In some embodiments, the proximal cuff is axially longer than the distal cuff.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic perspective view of a laryngeal tube, according to certain embodiments; and

FIG. 2 is a schematic cross-sectional view taken along a line A-A′ of the laryngeal tube of FIG. 1, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values there between.

Aspects of the present disclosure are directed to a laryngeal tube used in the larynx region of a patient during surgery and/or microsurgery to facilitate simultaneous ventilation and anesthesia. The laryngeal tube is preferably made of a non-rigid thermoplastic material such as polyvinyl chloride and includes a proximal cuff to secure the pharyngal area of the patient and a distal cuff to prevent oxygen from entering the stomach, and thereby to prevent stomach insufflation in case the laryngeal tube is inserted to the esophagus. The laryngeal tube further includes a suction lumen to reduce the risk of aspiration, a pharyngal lumen to provide ventilation capacities, and cuff inflation lines to communicate with the proximal cuff and the distal cuff. The laryngeal tube further includes a radiopaque cover through which the cuff inflation lines extend along with self-sealing valves and pilot balloons.

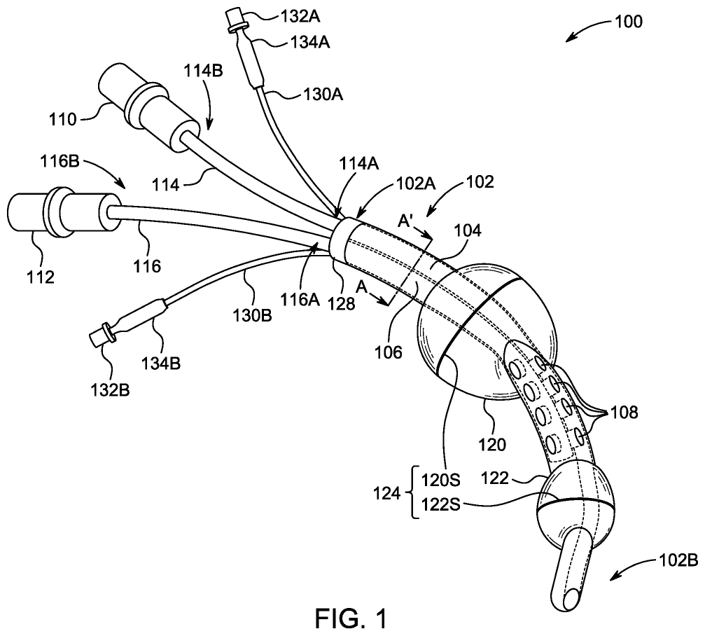

Referring to FIG. 1, a schematic perspective view of a laryngeal tube 100 is illustrated, according to an embodiment of the present disclosure. The laryngeal tube 100 includes an elongated body 102 having a proximal end 102A configured to be placed outside the mouth of a patient and a distal end 102B configured to be placed in the esophagus of the patient. The distal end 102B extends from the proximal end 102A of the elongated body 102 at an angle to facilitate easy insertion of the laryngeal tube 100 during an intubation procedure. The angle may be fixed, such as 30 degrees, 35 degrees, 40 degrees, or 45 degrees, or the angle may be adjustable. In an embodiment, a flexible tube that has a bendable element such as a copper tube or wire runs through the length of the elongated body 102 so that the copper wire permits the angle to be adjusted and hold its shape after adjustment. In an embodiment, the angled relation of the proximal end 102A and the distal end 102B can be described as an arc-shape, e.g., an arc along a single radius, as depicted in FIG. 1. The elongated body 102 of the laryngeal tube 100 may be made of thermoplastic material such as polyvinyl chloride or a flexible non-thermoplastic material such as silicone. In an embodiment, the elongated body 102 is a substantially cylindrical tube. The laryngeal tube 100 further includes a suction lumen 104 which extends along a length of the elongated body 102 and a pharyngal lumen 106 extending along a length of the elongated body 102. The suction lumen 104 may include an open end at the proximal end 102A of the laryngeal tube 100 and an open end at the distal end 102B of the elongated body 102. The suction lumen 104 is configured to reduce a risk of aspiration in a trachea of the patient. The pharyngal lumen 106 may include an open end at the proximal end 102A of the laryngeal tube 100 and has a plurality of pharyngal perforations 108 near the distal end 102B. In essence, the suction lumen 104 and pharyngal lumen 106 are disposed along the body 102 and are hollow tubes that pass through the elongated body 102. In an embodiment, the suction lumen 104 includes at least 4 pharyngal perforations 108, preferably at least 6, preferably at least 8, or at least 10. In an embodiment, the pharyngal lumen 106 includes at least 4 pharyngal perforations 108, preferably at least 6, preferably at least 8, or at least 10. In an embodiment, the pharyngal perforations 108 are substantially circular. In an embodiment, a diameter of the pharyngal perforations 108 is from 0.5 to 0.8 times the diameter of the elongated body 102, preferably from 0.55 to 0.75 times, preferably from 0.6 to 0.7 times, or 0.65 times the diameter of the elongated body 102. In an embodiment, the at least four pharyngal perforations 108 in the suction lumen 104 are axially aligned with the pharyngal lumen 106 along the length of the elongated body 102. The at least four pharyngal perforations 108 are preferably likewise axially aligned with the pharyngal lumen 106 along the length of the elongated body 102. In an embodiment, the at least four pharyngal perforations 108 in the suction lumen 104 are offset along the length of the elongated body 102 with the at least four pharyngal perforations 108 of the pharyngal lumen 106. In an embodiment, each of the suction lumen 104 and pharyngal lumen 106 have four pharyngal perforations 108. In an embodiment, each perforation is on an upper side of the suction lumen 104 extending through the lumen to a lower side, e.g., passes through the lumen wall on a front side to an opposite side of the lumen. In an embodiment, the perforation on the lower side of the lumen is not substantially circular. As used herein, the term lower side refers to the lower surface of the suction lumen 104 relative to the elongated body 102, e.g., an interior surface disposed towards the axis of the elongated body. As used herein, the term upper side refers to the upper surface of the suction lumen 104 relative to the elongated body 102, meaning the upper surface faces outward relative to the axis of the elongated body 102. In an embodiment, when the lumen is straightened (e.g., when inserted into the esophagus of a patient), the perforation on the upper surface/side of the lumen goes from a semi-circle shape to a circular shape at the upper side of the lumen. In an embodiment, when the lumen is straightened the perforation on the lower surface/side of the lumen goes from an oblong shape to a substantially circular shape.

The suction lumen 104 disposed in the elongated body 102 of the laryngeal tube 100 is in fluid communication with a suction conduit 114. The suction lumen 104 ends at the proximal end 102A of the elongated body 102 and then the suction conduit 114 begins, and the two are connected to form an uninterrupted channel. Particularly, the suction conduit 114 may include a first end 114A configured to be in fluid communication with the open end of the suction lumen 104 at the proximal end 102A of the elongated body 102, and a second end 114B configured to fixedly attach with an adapter 110, which is otherwise referred to as ‘the suction adapter 110’. The suction adapter 110 is configured to connect the suction lumen 104 to an air handling device (not shown) such that the suction lumen 104 reduces the risk of aspiration in the trachea of the patient. In an embodiment, the air handling device is a medical syringe or pump. An opening of the suction conduit 114 at the second end 114B thereof is alternatively referred to as ‘the suction port 114B’. The suction lumen 104 defined in the elongated body 102 of the laryngeal tube 100 and the suction conduit 114 may be together referred to as ‘the suction lumen 104’ unless otherwise specifically mentioned. The suction adapter 110 is fixedly attached to the suction lumen 104 by the suction port 114B. In some embodiments, the suction lumen 104 is fixedly attached to a suction connector or a jet ventilation tube. In some embodiments, the suction connector or the jet ventilation tube may be connected to the suction lumen 104 via the suction adapter 110. In some embodiments, the suction adapter 110 has a length in a longest dimension of from 10 mm to 50 mm, preferably from 20 mm to 40 mm, or 30 mm. In an embodiment, the suction conduit 114 is substantially cylindrical. In an embodiment, a diameter of the suction conduit 114 is from 0.4 to 0.7 times greater than the diameter of the elongated body 102, preferably from 0.45 to 0.65 time greater, preferably from 0.5 to 0.6 times greater, or 0.55 times greater. In an embodiment, the suction adapter 110 is substantially cylindrical. In an embodiment, the diameter of the elongated body 102 is the same diameter of the suction adapter 110. In an embodiment, the diameter of the suction adaptor 110 is from 0.7 to 0.9 times greater than the diameter of the elongated body 102, preferably 0.8 times greater. In an embodiment, the suction conduit 114 exits the elongated body 102 at the proximal end 102A of the elongated body 102 near the first end 114A.

Similarly, the pharyngal lumen 106 defined in the elongated body 102 of the laryngeal tube 100 is in fluid communication with a pharyngal conduit 116. Particularly, the pharyngal conduit 116 may include a first end 116A configured to fluidly communicate with the open end of the pharyngal lumen 106 defined at the proximal end 102A of the elongated body 102 and a second end 116B configured to fixedly attach with a pharyngal adapter 112. The pharyngal adapter 112 is configured to fluidly communicate the pharyngal lumen 106 with an air handling device (not shown). In an embodiment, the air handling device is a medical syringe or a pump. The pharyngal lumen 106 defined in the elongated body 102 of the laryngeal tube 100 and the pharyngal conduit 116 may be together referred to as ‘the pharyngal lumen 106’ unless otherwise specifically mentioned. As such, the pharyngal adapter 112 is fixedly attached to the pharyngal lumen 106. In some embodiments, the pharyngal adapter 112 has a length in a longest dimension of from 10 mm to 50 mm, preferably from 20 mm to 40 mm, or 30 mm. In an embodiment, the pharyngal adapter 112 has a length that is from 1.1 to 1.3 times greater than the length of the suction adapter 110, preferably 1.2 times greater. In an embodiment, the suction adapter 110 has a length that is from 1.1 to 1.3 times greater than the length of the pharyngal adapter 112, preferably 1.2 times greater. In an embodiment, the pharyngal conduit 116 is substantially cylindrical. In an embodiment, a diameter of the pharyngal conduit 116 is from 0.4 to 0.7 times greater than the diameter of the elongated body 102, preferably from 0.45 to 0.65 time greater, preferably from 0.5 to 0.6 times greater, or 0.55 times greater. In an embodiment, the diameter of the pharyngal conduit 116 is from 1.1 to 1.3 times greater than the diameter of the suction conduit 114, preferably 1.2 times greater. In an embodiment, the diameter of the suction conduit 114 is from 1.1 to 1.3 times greater than the diameter of the pharyngal conduit 116, preferably 1.2 times greater. In an embodiment, the pharyngal adapter 112 is substantially cylindrical. In an embodiment, the diameter of the elongated body 102 is the same diameter of the pharyngal adapter 112. In an embodiment, the diameter of the pharyngal adaptor 112 is from 0.7 to 0.9 times greater than the diameter of the elongated body 102, preferably 0.8 times greater. In an embodiment, the pharyngal conduit 116 exits the elongated body 102 at the proximal end 102A of the elongated body 102 near the first end 116A.

In some embodiments, each of the suction conduit 114 and the pharyngal conduit 116 connects with one of three different adapters, such as a 15 mm adaptor, the suction connector, or the jet ventilation tube in case the laryngeal tube 100 has high resistance. The 15 mm adaptor, which is used to connect the laryngeal tube 100 to a mechanical ventilator or an ambulatory bag, can be placed in the suction port 114B in case of tracheal ventilation. If the laryngeal tube 100 experiences high resistance, then the jet ventilation tube may be used. In an embodiment, the suction connector and jet ventilation tube are substantially cylindrical. In an embodiment, a diameter of the suction connector is the same diameter as the jet ventilation tube. In an embodiment, a length of both the suction connector and the jet ventilation tube is the same length as the adaptor.

The laryngeal tube 100 further includes a proximal cuff (e.g., pharyngal balloon) 120 disposed approximately midway along the length of the elongated body 102 and configured to secure a pharyngal area of the patient. Herein, the midway of the elongated body 102 may refer to a point equidistant from the distal end 102B and the proximal end 102A, or may be biased to one end by an amount of 5%, 10%, 20% or 30% of the entire length of the elongated body. The laryngeal tube 100 further includes a distal cuff 122 disposed near the distal end 102B of the elongated body 102 and configured to prevent oxygen escape into a stomach of the patient. In an embodiment, the at least eight pharyngal perforations 108 are disposed between the distal cuff 122 and the proximal cuff 120. The proximal cuff 120 and the distal cuff 122 are inflated or deflated by an external pumping device during the intubation procedure. In an embodiment, the proximal cuff 120 operates at a pressure of from 20 to 50 cm H₂O, preferably from 30 to 40 cm H₂O, or 35 cm H₂O to properly secure the pharyngal area of the patient and the distal cuff 122 operates at a pressure of from 20 to 60 cm H₂O, preferably from 30 to 50 cm H₂O, or 40 cm H₂O to prevent oxygen escape into the stomach of the patient.

In an embodiment, the external pumping device is a syringe or a pump. The external pumping device may be used to operate the proximal cuff 120 at a pressure between 20 and 50 cm H₂O and the distal cuff 122 at a pressure between 20 and 60 cm H₂O with the help of a pressure regulator. In an embodiment, each of the distal cuff 122 and proximal cuff 120 are preferably fabricated of polyvinyl chloride or a silicone. In an embodiment, an outer surface of each of the proximal cuff 120 and the distal cuff 122 includes an adhesive to help adhere to an airway, such as an adhesive tape or tube fastener. A capacity, or volume at full expansion, of the proximal cuff 120 is greater than a capacity of the distal cuff 122, as such the proximal cuff 120 is axially longer than the distal cuff 122. In an embodiment, the capacity of the proximal cuff 120 is from 1.2 to 1.6 times greater than the capacity of the distal cuff 122, preferably from 1.3 to 1.5 time greater, or 1.4 times greater. In an embodiment, the proximal cuff 120 accommodates a volume of fluid ranging from 60 milliliters (mL) to 100 mL, preferably from 70 mL to 90 mL, or 80 mL and the distal cuff 122 accommodates a volume of fluid ranging from 60 milliliters (mL) to 80 mL, or 70 mL.

In some embodiments, the suction lumen 104 has a first diameter at a distal end of the suction lumen to the suction port and a second diameter at a proximal end of the suction lumen to the suction port, as shown in FIG. 1. In an embodiment, the suction lumen 104 has a second diameter that is from 0.5 to 0.8 times the first diameter of the suction lumen, preferably from 0.55 to 0.75, preferably from 0.6 to 0.7, or 0.65. In an embodiment, the first diameter and the second diameter are equal. If the laryngeal tube 100 is inserted to the trachea, then the pharyngal lumen 106 can be used for suction and the suction lumen 104 can be used for ventilation, which is done only if the tracheal intubation is confirmed.

During the intubation procedure, the proximal cuff 120 and the distal cuff 122 may be in a deflated condition. Upon inserting the laryngeal tube 100 through the mouth of the patient, the proximal cuff 120 may be inflated to firmly position the laryngeal tube 100 by engaging the inflated proximal cuff 120 with the pharyngal area of the patient and the distal cuff 122 may be inflated to firmly position the distal end 102B of the laryngeal tube 100 in the esophagus of the patient. In an embodiment, a lubricant or adhesive is used on the outer surface of each of the proximal cuff 120 and distal cuff 122 to help secure the individual cuff to the respective area of the patient, Further, the proximal cuff 120 and the distal cuff 122 act as a seal in the pharyngal area and the esophagus of the patient, respectively, to prevent escape of oxygen therethrough. In some embodiments, the proximal cuff 120 includes a proximal cuff seal 120S and the distal cuff 122 includes a distal cuff seal 122S. The proximal cuff seal 120S and the distal cuff seal 122S are collectively referred to as ‘the cuff seals 124’ and individually referred to as ‘the cuff seal 124’ unless otherwise specifically mentioned. The cuff seal 124 may be made of a thermoplastic material such as polyvinyl chloride. In an embodiment, the cuff seal 124 is adjusted to wrap tightly over each of the proximal cuff 120 and distal cuff 122 when either cuff is inflated. In essence, the cuff seal 124 is sized to fit over each cuff at full expansion, rather than full deflation. Further, the cuff seal 124 may be flexible to attach with an outer surface of a material of the proximal cuff 120 and the distal cuff 122. In an embodiment, the cuff seal 124 may be an individual component attached to the material of the proximal cuff 120 and the distal cuff 122 using an adhesive. In another embodiment, the cuff seal 124 may be manufactured along with the proximal cuff 120 and the distal cuff 122 to form as a single integrated component. In another embodiment, the cuff seal 124 is removable from the proximal cuff 120 and the distal cuff 122 after operation is complete.

When the laryngeal tube 100 is placed in the mouth of the patient, the cuff seal 124 of each of the proximal cuff 120 and the distal cuff 122 may provide additional sealing capabilities to prevent oxygen flowing to the stomach and leakage of any other gas therethrough.

The laryngeal tube 100 further includes an optional radiopaque cover 128 having a first end disposed at the proximal end 102A of the laryngeal tube 100 and a second end extending through the proximal cuff 120 and disposed at the distal cuff 122 or distal end 102B. As used herein, the term “radiopaque,” refers to being opaque/impenetrable to x-rays. Particularly, the radiopaque cover 128 preferably connects the pharyngal lumen 106 to the proximal cuff 120 and distal cuff 122. The radiopaque cover 128 further includes a plurality of perforations corresponding to the plurality of pharyngal perforations 108 defined in the elongated body 102 of the laryngeal tube 100 to communicate with the pharyngal lumen 106. In an embodiment, the radiopaque cover 128 includes at least 8 pharyngal perforations 108, at least 12 pharyngal perforations 108, at least 16 pharyngal perforations 108, or at least 20 pharyngal perforations 108. In an embodiment, the radiopaque cover 128 fully covers the pharyngal lumen 106 and the suction lumen 104. That is, the suction lumen 104 and pharyngal lumen 106 run internally through the elongated body 102 which is covered by the radiopaque cover 128. In essence, the radiopaque cover 128 is a coating/cover preferably on the entire outer surface of the pharyngal lumen 106 and the suction lumen 104. In an embodiment, the radiopaque cover comprises one or more radiopaque fillers, such as barium sulfate, bismuth trioxide, bismuth oxychloride, bismuth subcarbonate, tungsten, iodine, tantalum, gold, or a combination thereof. In an embodiment, the radiopaque cover 128 is positioned between the suction lumen 104 and the pharyngal lumen 106 as a partition between the lumens. In an embodiment, the radiopaque cover 128 extends from the proximal end 102A of the elongated body 102 to the proximal cuff 120. In an embodiment, the radiopaque cover 128 surrounds a periphery of the suction lumen 104 and the pharyngal lumen 106. In an embodiment, a diameter of the radiopaque cover 128 is from 1.75 to 2.25 times greater than a diameter of the suction lumen 104, preferably 1.8 to 2.2 times greater, preferably 1.9 to 2.1 times greater, or 2 times greater. In an embodiment, a diameter of the radiopaque cover 128 is from 1.75 to 2.25 times greater than a diameter of the pharyngal lumen 106, preferably 1.8 to 2.2 times greater, preferably 1.9 to 2.1 times greater, or 2 times greater.

The laryngeal tube 100 further includes a first cuff inflation line 130A configured to inflate or deflate the proximal cuff 120 and a second cuff inflation line 130B configured to inflate or deflate the distal cuff 122. The first cuff inflation line 130A and the second cuff inflation line 130B are collectively referred to as ‘the cuff inflation lines 130’ and individually referred to as ‘the cuff inflation line 130’ unless otherwise specifically mentioned. In some embodiments, the laryngeal tube 100 may include one cuff inflation line 130 for inflating or deflating the proximal cuff 120 and the distal cuff 122. Each of the first cuff inflation line 130A and the second cuff inflation line 130B may include a first end configured to engage with an external pumping device and a second end configured to fluidly couple with the proximal end 102A of the elongated body 102. In an embodiment, each of the first cuff inflation line 130A and second cuff inflation line 130B are substantially cylindrical tubes. In an embodiment, the diameters of each of the first cuff inflation line 130A and second cuff inflation line 130B are from 0.3 to 0.7 times greater than the diameters of the suction conduit 114 and pharyngal conduit 116, preferably from 0.4 to 0.6 times greater, or 0.5 times greater. In an embodiment, the lengths of each of the first cuff inflation line 130A and second cuff inflation line 130B are from 0.5 to 0.8 times greater than the length of the elongated body 102, preferably from 0.55 to 0.75 times greater, preferably from 0.6 to 0.7 times greater, or 0.65 times greater. The first cuff inflation line 130A includes a first self-sealing valve 132A and the second cuff inflation line 130B includes a second self-sealing valve 132B. The first self-sealing valve 132A and the second self-sealing valve 132B are collectively referred to as ‘the self-sealing valves 132’ and individually referred to as ‘the self-sealing valve 132’ unless otherwise specifically mentioned. Each of the first self-sealing valve 132A and the second self-sealing valve 132B is connected to the first end of each of the first cuff inflation line 130A and the second cuff inflation line 130B, respectively. Further, each of the first self-sealing valve 132A and the second self-sealing valve 132B surrounds a periphery of each of the first cuff inflation line 130A and the second cuff inflation line 130B, respectively. In an embodiment, the self-sealing valves 132 is a one-way valve. The first cuff inflation line 130B further includes a first pilot balloon 134A and the second cuff inflation line 130B includes a second pilot balloon 134B. The first pilot balloon 134A and the second pilot balloon 134B are collectively referred to as ‘the pilot balloons 134’ and individually referred to as ‘the pilot balloon 134’ unless otherwise specifically mentioned. In an embodiment, the first pilot balloon 134A is positioned between the first cuff inflation line 130A and first self-sealing valve 132A. In an embodiment, the second pilot balloon 134B is positioned between the second cuff inflation line 130B and second self-sealing valve 132B. Each of the first pilot balloon 134A and the second pilot balloon 134B is inflated while inflating the proximal cuff 120 and the distal cuff 122 and helps to indicate inflated status of the proximal cuff 120 and the distal cuff 122. In an embodiment, each of the first cuff inflation line 130A and the second cuff inflation line 130B is made of polyvinyl chloride (PVC). In an embodiment, the pilot balloons 134 are made of polyvinyl chloride. In an embodiment, the self-sealing valves 132 include a plurality of grooves to help the operator open and close the valve as necessitated. In an embodiment, there are from 4 to 10 grooves, preferably 6 to 8 grooves, or 7 grooves for each self-sealing valve 132. The grooves function as a friction source for the user to touch to help them open and close the valves. In essence, the grooves for self-sealing valves 132 help the user twist the valve manually during intubation without taking surgical gloves off. In an embodiment, the first self-sealing valve 132A, a first pilot balloon 134A, and a first cuff inflation line 130A are collected into a first tube, as depicted in FIG. 1. In an embodiment, the first tube is made of

PVC. In an embodiment, the first tube extends to the proximal end 102A of the elongated body 102. In an embodiment, a second self-sealing valve 132B, a second pilot balloon 134B, and a second cuff inflation line 130B are collected into a second tube as depicted in FIG. 1. In an embodiment, the second tube is made of PVC. In an embodiment, the second tube extends to the proximal end 102A of the elongated body 102. In an embodiment, the first and second tube have the same diameter. In an embodiment, the first tube is from 0.7 to 0.9 times greater than a diameter of the second tube, preferably 0.8 times greater. In an embodiment, the second tube is from 0.7 to 0.9 times greater than a diameter of the first tube, preferably 0.8 times greater. In an embodiment, the pharyngal lumen 106 has a first diameter from a distal end of the pharyngal lumen 106 to the second pilot balloon 134B and a second diameter from a proximal end of the pharyngal lumen 106 to the second pilot balloon 134B. In an embodiment, the second diameter is less than the first diameter. In an embodiment, the second diameter is equal to the first diameter. In an embodiment, the second diameter provides the pharyngal lumen volume of from 50% to 70% the volume of the second pilot balloon 134B, preferably from 55% to 65%, or 60%. In an embodiment, the first cuff inflation line 130A is continuous and exits the elongated body 102 at the proximal end 102A of the elongated body 102 near the first end 114A. In an embodiment, the second cuff inflation line 130B is continuous and exits the elongated body 102 at the proximal end 102A of the elongated body 102 near the first end 116A.

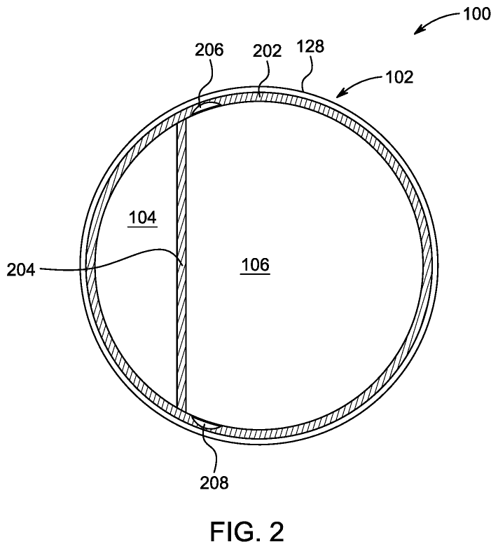

Referring to FIG. 2, a schematic cross-sectional view taken along a line A-A′ of the laryngeal tube 100 is illustrated, according to an embodiment. The laryngeal tube 100 includes the elongated body 102 having an outer wall 202 defining an inflated circular cross-section. In some embodiments, the elongated body 102 may have an elliptical cross-section. As shown in FIG. 2, the elongated body 102 may include a separator wall 204 for defining the suction lumen 104 and the pharyngal lumen 106. In an embodiment, the separator wall 204 extends down the middle diameter axis of the outer wall 202 equidistant from opposite sides of the outer wall 202. In an embodiment, the separator wall 204 is offset towards either side of the middle diameter axis. In a preferable embodiment, the pharyngal lumen 106 may have a cross-sectional area greater than a cross-sectional area of the suction lumen 104. In an embodiment, a height of the separator wall 204 is from 0.6 to 0.9 times an inner diameter of the elongated body 102, 0.6 to 0.7 times the inner diameter of the elongated body 102, or about 0.7 times the inner diameter of the elongated body 102. The plurality of pharyngal perforations 108 may be defined in a portion of the outer wall 202 that defines the pharyngal lumen 106 with the separator wall 204.

Any in a preferred embodiment, the separator wall 204 is made of a material that is different from the material of the outer wall 202. This difference may be in composition and is preferably a difference in properties. In order to facilitate insertion into a patient the material forming the outer wall 202 is preferably flexible and elastic such that the cavities defining the suction lumen 104 and then pharyngal lumen 106 may collapse or partially collapse during insertion into the esophagus of a patient. The separator wall 204 is preferably of greater stiffness than the outer wall 202. In this respect the separator wall 204 may have a greater thickness and/or be made of a material having greater rigidity than the material of the outer wall 202. The separator wall is preferably a plasticized PVC having a flexural modulus that is greater than the flexural modulus of the outer wall 202, e.g., a flexural modulus of the separator wall is from 1.9 to 2.4 GPa, 2.0 to 2.3 GPa or about 2.2 GPa according to ASTM D790-17.

Although shown as a flat separator wall 204 in FIG. 2, the separator wall 204 may have or adopt a pre-formed shape that is preferably semi-circular. The radius of the semi-circular form of the separator wall 204 is preferably greater than the radius of the circular outer wall 202. In this form the separator wall 204 provides sufficient stiffness to aid during insertion, and resists folding, kinking or bending both upwards and downwards from the plane of the separator wall 204 concurrently. Slight compression of the semi-circular shape favors bending inwardly towards the radius of the semi-circle and resists bending out of plane. Moreover, the semicircular form permits compression of the separation wall 204 to further ease insertion.

In one embodiment, a first passage 206 may be defined in the outer wall 202 of the elongated body 102 and configured to fluidly communicate with the proximal cuff 120. The first passage 206 may be further connected to the first cuff inflation line 130A at the proximal end 102A of the elongated body 102. The first cuff inflation line 130A and the first passage 206 may be together referred to as ‘the first cuff inflation line 130A’ unless otherwise specifically mentioned. In an embodiment, the first passage 206 is substantially circular or semicircular. In an embodiment, a diameter or cross-sectional area of the elongated body 102 is from 10 to 50 times a diameter or cross-sectional area of the first passage 206, preferably from 20 to 40 times, or 30 times. Similarly, a second passage 208 may be defined in the outer wall 202 of the elongated body 102 and configured to fluidly communicate with the distal cuff 122. The second passage 208 may be further connected to the second cuff inflation line 130B at the proximal end 102A of the elongated body 102. The second cuff inflation line 130B and the second passage 208 may be together referred to as ‘the second cuff inflation line 130B’ unless otherwise specifically mentioned. In an embodiment, the second passage 208 is substantially circular. In an embodiment, a diameter or cross-sectional area of the elongated body 102 is from 10 to 50 times greater than a diameter or cross-sectional area of the second passage 208, preferably from 20 to 40 times greater, or 30 times greater. In an embodiment, the diameter of the first passage 206 is greater than the diameter of the second passage 208. The first and second passages are preferably opposite one another around the circumference of the elongated body 102.

In an alternate embodiment, the suction conduit 114 and the pharyngal conduit 116 extend through the interior of the radiopaque cover 128. The radiopaque cover 128 may be a hollow cylindrical body having an inner diameter greater than an outer diameter of each of the suction port 114B, the first and second cuff inflation lines 130A, 130B, the first and second self-sealing valves 132A, 132B, and the first and second pilot balloons 134A, 134B. Particularly, the inner diameter of the radiopaque cover 128 is from 1.1 to 1.4 times greater, preferably 1.25 times greater than the outer diameter of the suction port 114B, 1.1 to 1.4 times greater, preferably 1.25 times greater than the outer diameter of each of the first and second cuff inflation lines 130A, 130B, 1.1 to 1.4 times greater, preferably 1.25 times greater than the outer diameter of each of the first and second self-sealing valves 132A, 132B, and 1.2 to 1.5 times greater, preferably 1.35 times greater than the outer diameter of each of the first and second pilot balloons 134A, 134B. In an embodiment, the radiopaque cover 128 extends over the entire length elongated body 102.

According to the present disclosure, the elongated body 102 of the laryngeal tube 100 is preferably made of polyvinyl chloride and includes the proximal cuff 120 and the distal cuff 122. In case the laryngeal tube 100 is inserted into the trachea of the patient, the proximal cuff 120 is ignored and the distal cuff 122 is used to prevent aspiration. In case of tracheal intubation, the proximal and distal cuffs 120, 122 serve to secure the laryngeal tube 100, prevent leak of oxygen 10 from the lung, and prevent aspiration in case the patient vomits. Both the proximal and distal cuffs 120, 122 are low-pressure and high-volume cuffs.

Numerous modifications and variations of the present disclosure are possible in light of the above teachings. It is therefore to be understood that within the scope of the appended claims, the invention may be practiced otherwise than as specifically described herein.

1: A laryngeal tube comprising:
a first tube comprising a first self-sealing valve, a first pilot balloon, and a first cuff inflation line;
a second tube comprising a second self-sealing valve, a second pilot balloon, and a second cuff inflation line;
a suction lumen;
a pharyngal lumen;
a suction port;
a radiopaque cover; and
a proximal cuff and a distal cuff, wherein the proximal cuff is configured to secure a pharyngal area of a patient and the distal cuff is configured to prevent oxygen escape into a stomach of the patient; and
wherein the first self-sealing valve surrounds a periphery of the first cuff inflation line and the second self-sealing valve surrounds a periphery of the second cuff inflation line;
wherein the first tube is fluidly connected to the proximal cuff and the second tube is fluidly connected to the second cuff;
wherein the suction lumen, the pharyngal lumen, the first tube, and the second tube are at least partially disposed in the elongated body and at least partially covered by the radiopaque cover; and
wherein the suction lumen and the pharyngal lumen have a common outer wall inside the elongated body and a common separator wall.

2: The laryngeal tube of claim 1, wherein the cuff inflation line is made of polyvinyl chloride (PVC).

3: The laryngeal tube of claim 1, wherein the separator wall has a semicircular profile and the outer wall defines a tube having a circular cross section, wherein the radius of the semicircular profile of the separator wall is greater than the radius of the circular cross section of the outer wall.

4: The laryngeal tube of claim 3, wherein the separator wall has a height that is 0.5-0.9 times the inner diameter of the outer wall.

5: The laryngeal tube of claim 1, wherein the radiopaque cover comprises at least eight pharyngal perforations.

6: The laryngeal tube of claim 1, wherein the proximal cuff operates at a pressure of from 20 and 50 cm H₂O to properly secure the pharyngal area of the patient.

7: The laryngeal tube of claim 1, wherein the distal cuff operates at a pressure of from 20 and 60 cm H₂O to prevent oxygen escape into the stomach of the patient.

8: The laryngeal tube of claim 1, wherein the proximal cuff accommodates a volume of fluid ranging from 60 milliliters (mL) to 100 mL.

9: The laryngeal tube of claim 1, wherein the distal cuff accommodates a volume of fluid ranging from 60 milliliters (mL) to 80 mL.

10: The laryngeal tube of claim 9, wherein the suction lumen has a first diameter at a distal end of the suction lumen and a second diameter at a proximal end of the suction lumen.

11: The laryngeal tube of claim 9, wherein the pharyngal lumen has a first diameter at a distal end of the pharyngal lumen and a second diameter at a proximal end of the pharyngal lumen; and
wherein the second diameter is less than the first diameter.

12: The laryngeal tube of claim 3, further comprising an adapter fixedly attached to the suction lumen by the suction port.

13: The laryngeal tube of claim 3, further comprising a suction connector or a jet ventilation tube attached to the suction lumen.

14: The laryngeal tube of claim 1, wherein an inner diameter of the radiopaque cover is from 1.1 to 1.4 times greater than an outer diameter of the suction port.

15: The laryngeal tube of claim 1, wherein an inner diameter of the radiopaque cover is from 1.1 to 1.4 times greater than an outer diameter of the cuff inflation line.

16: The laryngeal tube of claim 1, wherein an inner diameter of the radiopaque cover is from 1.1 to 1.4 times greater than an outer diameter of the self-sealing valve.

17: The laryngeal tube of claim 1, wherein an inner diameter of the radiopaque cover is from 1.2 to 1.5 times greater than an outer diameter of the pilot balloon.

18: The laryngeal tube of claim 1, wherein the proximal cuff comprises a proximal cuff seal.

19: The laryngeal tube of claim 1, wherein the distal cuff comprises a distal cuff seal.

20: The laryngeal tube of claim 1, wherein the proximal cuff is axially longer than the distal cuff.
